(19) United States
(12) Patent Application Publication
(10) Pub. No.: US 2023/0238144 A1
(43) Pub. Date: Jul. 27, 2023

(54) STROKE EXAMINATION SYSTEM, STROKE EXAMINATION METHOD, AND RECORDING MEDIUM

(71) Applicant: Panasonic Intellectual Property Management Co., Ltd., Osaka (JP)

(72) Inventors: Tomoki OGAWA, Osaka (JP); Kazuhiro IDE, Osaka (JP); Kohsuke YAMAMOTO, Osaka (JP)

(21) Appl. No.: 18/009,550

(22) PCT Filed: Aug. 5, 2021
(86) PCT No.: PCT/JP2021/029165

§ 371 Date: Dec. 9, 2022

Related U.S. Application Data
(60) Provisional application No. 63/061,691, filed on Aug. 5, 2020.

(51) Int. Cl. G16H 50/30; G16H 10/60; G16H 50/20

(52) U.S. Cl. CPC G16H 50/30; G16H 10/60; G16H 50/20

(57) ABSTRACT

A stroke examination system which conducts an examination for a sign of stroke in a subject includes: an obtainer which obtains profile information relating to a brain disorder of the subject; a determiner which determines a priority of each of a plurality of examination items for stroke, based on the profile information obtained; a plurality of examiners which conducts examinations of the plurality of the examination items in a descending order of the priority determined; and a diagnosis unit which outputs diagnostic information relating to the sign of stroke in the subject based on an examination result obtained by the plurality of the examination items.

### CROSS-REFERENCE OF RELATED APPLICATIONS

This application is the U.S. National Phase under 35 U.S.C. § 371 of International Patent Application No. PCT/JP2021/029165, filed on Aug. 5, 2021, which in turn claims the benefit of U.S. Application No. 63/061,691, filed on Aug. 5, 2020, the entire disclosures of which Applications are incorporated by reference herein.

### TECHNICAL FIELD

The present disclosure relates to a stroke examination system, a stroke examination method, and a recording medium which conduct an examination for signs of stroke in a subject.

### BACKGROUND ART

It is known that at the time of a stroke, a prompt treatment increases the probability of recovery without major after-effects. Hence, when a stroke is suspected to have occurred, it is desirable that an examination for signs of stroke can be quickly conducted. For example, a stroke detection method has been developed which is capable of quickly conducting an examination for signs of stroke by conducting a simple examination using an information terminal, such as a smartphone, which is recently owned and carried by many people (see, for example, Patent Literature (PTL) 1).

### CITATION LIST

### Patent Literature

[PTL 1] International Application Publication No. WO2018/053521

### SUMMARY OF INVENTION

### Technical Problem

In view of conducting an appropriate examination, the stroke detection method and the like as disclosed in PTL 1 may be insufficient.

The present disclosure has been conceived in view of the above. An object of the present disclosure is to provide a stroke examination system and the like which is capable of conducting an appropriate examination.

### Solution to Problem

In order to achieve the object described above, one aspect of a stroke examination system according to the present disclosure is a stroke examination system which conducts an examination for a sign of stroke in a subject. The stroke examination system includes: an obtainer which obtains profile information relating to a brain disorder of the subject; a determiner which determines a priority of each of a plurality of examination items for stroke based on the profile information obtained; a plurality of examiners which conduct examinations of the plurality of the examination items in a descending order of the priority determined; and a diagnosis unit which outputs diagnostic information relating to the sign of stroke in the subject based on an examination result obtained by the plurality of examiners.

Moreover, one aspect of a stroke examination method according to the present disclosure includes: obtaining profile information relating to a medical history of a brain disorder of a subject; determining a priority of each of a plurality of examination items for stroke based on the profile information obtained; and conducting an examination of each of the plurality of examination items in a descending order of the priority determined.

General and specific aspects disclosed above may be implemented using a system, a device, an integrated circuit, a computer program, a computer-readable recording medium such as a CD-ROM, or any combination of systems, devices, integrated circuits, computer programs, or recording media.

### Advantageous Effects of Invention

The present disclosure provides a stroke examination system and the like which is capable of conducting an appropriate examination.

### BRIEF DESCRIPTION OF DRAWINGS

FIG. 1 schematically illustrates one example of a configuration of a stroke examination system according to an embodiment.

FIG. 2 is a block diagram which illustrates a functional configuration of the stroke examination system according to the embodiment.

FIG. 3 is a block diagram to contrast the functional configuration of the stroke examination system according to the embodiment with a functional configuration of a smartphone that is one implementation of a portable terminal device.

FIG. 4 is a diagram for illustrating a triaxial sensor and a triaxial angular velocity sensor included in the smartphone that is one implementation of the portable terminal device.

FIG. 5 is a flowchart which illustrates an example of an operation of the stroke examination system according to the embodiment.

FIG. 6 is a first diagram which illustrates one example of an operation screen of the stroke examination system according to the embodiment.

FIG. 7A is a second diagram which illustrates one example of an operation screen of the stroke examination system according to the embodiment.

FIG. 7B is a third diagram which illustrates one example of an operation screen of the stroke examination system according to the embodiment.

FIG. 8A is a fourth diagram which illustrates one example of an operation screen of the stroke examination system according to the embodiment.

FIG. 8B is a fifth diagram which illustrates one example of an operation screen of the stroke examination system according to the embodiment.

FIG. 9A is a sixth diagram which illustrates one example of an operation screen of the stroke examination system according to the embodiment.

FIG. 9B is a seventh diagram which illustrates one example of an operation screen of the stroke examination system according to the embodiment.

FIG. 10 illustrates one example of a neural network for examining facial paralysis in an implementation of the embodiment.

FIG. 11A illustrates one example of a neural network where consideration is given to an influence of rotation of a facial image caused when capturing the image for examining facial paralysis in an implementation of the embodiment.

FIG. 11B illustrates one example of a neural network where consideration is given to an influence of an inclination of an upper face (forehead part) and a lower face (chin part) in a facial image caused when capturing the image for examining facial paralysis in an implementation of the embodiment.

FIG. 12 illustrates one example of a process of correcting an inclination occurred when capturing the facial image of a subject in an implementation of the embodiment.

FIG. 13 illustrates a state in which the subject is capturing a facial image of oneself while holding the stroke examination device in an outstretched arm, and conducting examinations for Barre’s signs and facial paralysis simultaneously.

### DESCRIPTION OF EMBODIMENT

### Underlying Knowledge Forming the Basis of the Present Disclosure

In recent years, many people carry information terminals (such as smartphones, tablet terminals, and personal computers (PCs)) which are capable of performing high-performance information processing. As described in the background section above, an examination for signs of stroke has to be promptly conducted on a subject who is suspected to be having a stroke. In the case where there is suspicion of stroke in the subject, if a simple examination for signs of stroke can be conducted by the information terminal available at the time, appropriate measures can be taken rapidly according to the emergency at the time. Accordingly, as described in PTL 1, a stroke detection method and the like has been developed which is capable of quickly conducting an examination for signs of stroke by conducting a simple examination using an information terminal.

However, as described above, in the case where there is suspicion of stroke and the person with no previous knowledge operates the information terminal, necessary measures may be taken too late. Specifically, in a situation when every second counts, for example, time may be wasted by conducting examinations of a plurality of examination items sequentially, or performing incorrect operations caused by not understanding if operating instructions are for the subject or the operator when the subject and the operator are different.

In view of the above, the present disclosure provides a stroke examination system and the like which is capable of conducting examinations of a plurality of examination items in an appropriate order, and also providing an appropriate operating instruction based on whether or not the operator and the subject are the same in each of the examination items.

### Outline of Disclosure

An outline of the present disclosure is as described below.

A stroke examination system according to one aspect of the present disclosure is a stroke examination system which conducts an examination for a sign of stroke in a subject. The stroke examination system includes: an obtainer which obtains profile information relating to a brain disorder of the subject; a determiner which determines a priority of each of a plurality of examination items for stroke based on the profile information obtained; a plurality of examiners which conduct examinations of the plurality of the examination items in a descending order of the priority determined; and a diagnosis unit which outputs diagnostic information relating to the sign of stroke in the subject based on an examination result obtained by the plurality of examiners.

Such a stroke examination system determines the priority of each of the examination items based on the obtained profile information relating to the brain disorder of the subject, and conducts an examination of each examination item according to the priority. When the determined priority corresponds to, for example, usefulness of conducting an examination of the examination item for the subject, the examiner is capable of conducting the examination of each examination item in descending order of usefulness. Since a useful examination result can be obtained relatively early after the start of the examination, diagnosis and the like can be performed without waiting for the subsequent examination result. Accordingly, in terms of examination time, it is possible to appropriately conduct an examination for stroke that requires an emergency response.

Moreover, for example, it may be that the profile information relates to a medical history of the brain disorder of the subject.

With this, the priority of each examination item for stroke can be determined by using the information relating to the medical history of the brain disorder of the subject.

Moreover, for example, it may be that the plurality of examination items include at least one of an examination item for facial paralysis of the subject, an examination item for a Barre’s sign of the subject, an examination item for dysarthria of the subject, or an examination item for a gait abnormality of the subject.

With this, the plurality of examination items include different ones of the examination item for facial paralysis of the subject, the examination item for Barre’s signs of the subject, the examination item for dysarthria of the subject, and the examination item for gait abnormality. Accordingly, the priority can be determined for each of the examination items, and the examination can be conducted in the order of the determined priorities.

Moreover, for example, it may be that when the profile information includes a medical history involving a specific symptom of the subject, the determiner gives a higher priority to an examination item for the specific symptom of the subject among the plurality of examination items than a priority of an other examination item.

With this, when the subject has a medical history which likely causes to a specific symptom to develop, it is possible to increase the priority of the examination of the examination item that is conducted based on the presence or absence of the specific symptom. Accordingly, in the case where the recurrence of a specific symptom is assumed as a sign of stroke, the examination of the examination item can be preferentially conducted.

Moreover, for example, it may be that when the profile information includes a medical history involving a specific symptom of the subject, the determiner gives a lower priority to an examination item for the specific symptom of the subject among the plurality of examination items than a priority of an other examination item.

With this, in the case where the subject already has developed a specific symptom, it is possible to decrease the priority of the examination of the examination item that is conducted based on the presence or absence of the specific symptom. Accordingly, when it is not easy to determine whether or not the specific symptom is a newly appeared symptom as a sign of stroke, an examination of another examination item can be preferentially conducted.

Moreover, for example, it may be that when the profile information includes a medical history involving facial paralysis of the subject, the determiner gives a lower priority to an examination item for the facial paralysis of the subject among the plurality of examination items than a priority of an other examination item.

With this, in the case where the subject already has developed facial paralysis, it is possible to decrease the priority of the examination of the examination item that is conducted based on the presence or absence of the facial paralysis. Accordingly, when it is not easy to determine whether or not the facial paralysis is a newly appeared symptom as a sign of stroke, an examination of an examination item that is other than the facial paralysis can be preferentially conducted.

Moreover, for example, it may be that when the profile information includes a medical history involving a Barre’s sign of the subject, the determiner gives a lower priority of an examination item for the Barre’s sign among the plurality of examination items than a priority of an other examination item.

With this, in the case where the subject already has Barre’s signs occurring, it is possible to decrease the priority of the examination of the examination item that is conducted based on the presence or absence of the Barre’s signs. Accordingly, when it is not easy to determine whether or not the Barre’s signs are newly appeared symptoms as signs of stroke, an examination of an examination item other than the Barre’s signs can be preferentially conducted.

Moreover, for example, it may be that when the profile information includes a medical history involving dysarthria of the subject, the determiner gives a lower priority of an examination item for the dysarthria of the subject among the plurality of examination items than a priority of an other examination item.

With this, in the case where the subject already has developed dysarthria, it is possible to decrease the priority of the examination of the examination item that is conducted based on the presence or absence of the dysarthria. Accordingly, when it is not easy to determine whether or not the dysarthria is a newly appeared symptom as a sign of stroke, an examination of an examination item other than the dysarthria can be preferentially conducted.

Moreover, for example, it may be that when the profile information includes a medical history involving a gait abnormality of the subject, the determiner gives a lower priority of an examination item for the gait abnormality of the subject among the plurality of examination items than a priority of an other examination item.

With this, in the case where the subject already has developed gait abnormality, it is possible to decrease the priority of the examination of the examination item that is conducted based on the presence or absence of the gait abnormality. Accordingly, when it is not easy to determine whether or not the gait abnormality is a newly appeared symptom as a sign of stroke, an examination of an examination item other than the gait abnormality can be preferentially conducted.

Moreover, for example, it may be that the plurality of examiners include an examiner which does not conduct an examination of an examination item with a priority that is lower than or equal to a predetermined value.

With this, when the determined priority corresponds to, for example, usefulness of conducting an examination of the examination item for the subject, the examiner is capable of conducting the examination of each examination item in descending order of usefulness. Since a useful examination result can be obtained relatively early after the start of the examination, it is possible to omit subsequent examinations of the examination items with relatively lower priorities (with priorities lower than or equal to a predetermined value).

Moreover, for example, it may be that a storage is included which stores at least one of an onset history of the brain disorder of the subject or medical examination information including information relating to the brain disorder of the subject, and the obtainer obtains, as the profile information, at least one of the onset history or the medical examination information from the storage.

With this, at least one of the onset history or the medical examination information obtained from the storage can be used as the profile information.

Moreover, for example, it may be that an identifier is included which identifies the subject and outputs identification information, and the obtainer obtains the profile information corresponding to the identification information output.

With this, for the subject identified by the identifier, corresponding profile information can be obtained.

Moreover, for example, it may be that the profile information relates to a result of a preliminary examination conducted on the subject.

With this, information relating to the result of the preliminary examination conducted on the subject can be used as the profile information.

Moreover, a stroke examination method according to one aspect of the present disclosure includes: obtaining profile information relating to a medical history of a brain disorder of a subject; determining a priority of each of a plurality of examination items for stroke based on the profile information obtained; and conducting an examination of each of the plurality of examination items in a descending order of the priority determined.

Such a stroke examination method is capable of providing the same advantageous effects as the stroke examination system described above.

Moreover, a recording medium according to one aspect of the present disclosure is a non-transitory computer-readable recording medium for use in a computer. The recording medium has a computer program recorded thereon for causing the computer to execute the stroke examination method described above.

Such a recording medium is capable of providing the same advantageous effects as the stroke examination system described above by using a computer.

Moreover, a stroke examination system according to another aspect of the present disclosure is a stroke examination system which conducts an examination for a sign of stroke in a subject. The stroke examination system includes: an identifier which identifies whether or not an operator of the stroke examination system is the subject; an examiner which conducts an examination of a predetermined examination item for stroke, the examiner conducting the examination of the predetermined examination item in a first mode when the operator is identified as being the subject, the examiner conducting the examination of the predetermined item in a second mode that is different from the first mode when the operator is identified as not being the subject; and a diagnosis unit which outputs diagnostic information relating to the sign of stroke in the subject based on an examination result obtained by the examiner.

In such a stroke examination system, when the operator identified by the identifier is the subject, an examination of a predetermined examination item is conducted in a first mode where the subject operates the stroke examination system by oneself, and conducts the examination of the predetermined examination item. When the operator identified by the identifier is not the subject, an examination of a predetermined item is conducted in a second mode where the operator that is other than the subject operates the stroke examination system and conducts the examination of the predetermined examination item. This reduces the possibility that the examination time is wasted by performing incorrect operations caused by not understanding if operating instructions for conducting the examination are for the subject or the operator. Hence, in terms of examination time, it is possible to appropriately conduct an examination for stroke.

Hereinafter, an embodiment of the present disclosure will be described with reference to the drawings.

The following embodiment describes a general or specific example. The numerical values, shapes, materials, structural elements, the arrangement and connection of the structural elements, steps, order of the steps, etc., shown in the following embodiment are mere examples, and therefore do not limit the scope of the claims. Among the structural elements in the following embodiment, those not recited in any of the independent claims are described as optional elements.

The figures are not necessarily precise illustrations. In the figures, elements that are essentially the same share like reference signs. Accordingly, duplicate description thereof is omitted or simplified.

Moreover, in the present description, terms that describe a relationship between elements, such as “parallel”, terms that describe the shape of an element, such as “rectangular”, values, and value ranges are not limited to their precise meanings, but also include variations that fall within an essentially equivalent range, such as a degree of error of approximately a few percent.

### Embodiment

### [Configuration]

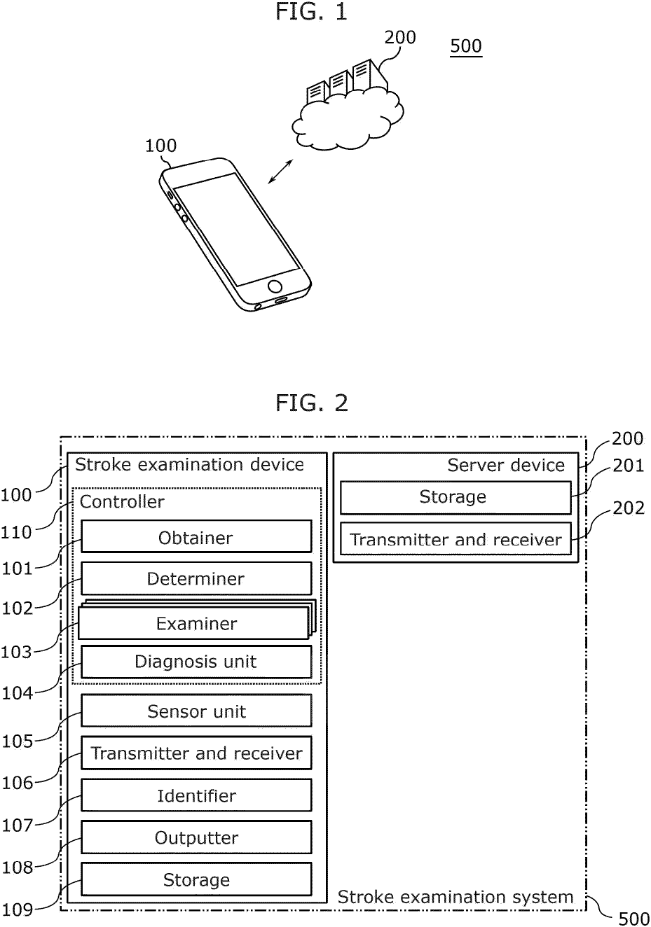

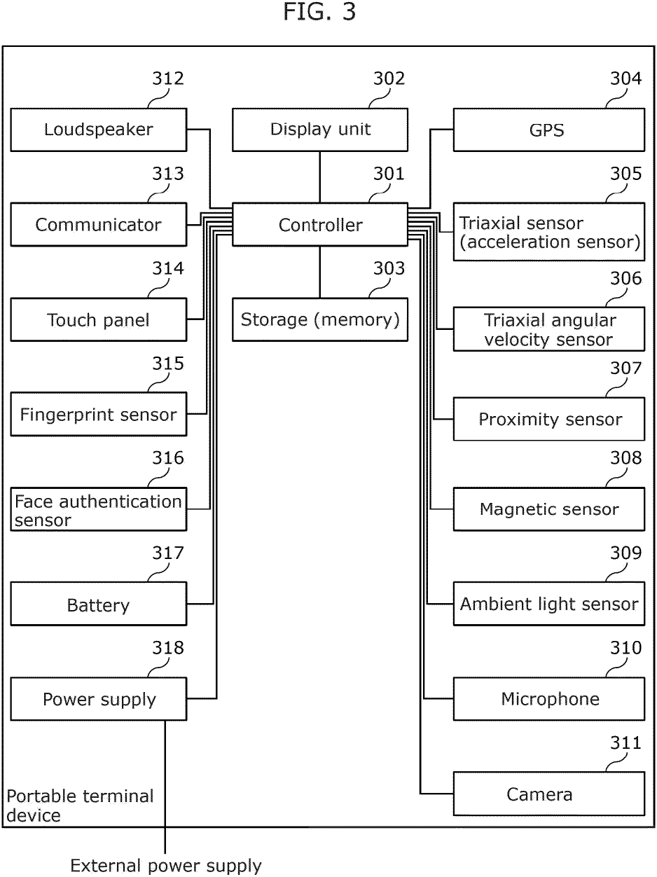

First, an outline of a stroke examination system according to an embodiment will be described with reference to FIG. 1 to FIG. 4. FIG. 1 schematically illustrates one example of a configuration of the stroke examination system according to the embodiment. FIG. 2 is a block diagram which illustrates a functional configuration of the stroke examination system according to the embodiment. FIG. 3 is a block diagram illustrating one example where the stroke examination system according to the embodiment is implemented by a portable terminal device such as a smartphone.

As illustrated in FIG. 1, stroke examination system 500 according to the present embodiment includes stroke examination device 100 that is realized by an information terminal, and server device 200.

Stroke examination device 100 includes sensors and the like corresponding to various examination items for examining signs of stroke. Stroke examination device 100 conducts an examination for signs of stroke of the subject making full use of the sensors and the like while sequentially giving necessary instructions for the examination to the operator of stroke examination system 500, that is, the operator of stroke examination device 100. Although stroke examination device 100 is realized by an information terminal, stroke examination device 100 may be realized by another device as long as the device includes a configuration for achieving the various functions described below. For example, stroke examination device 100 may be a dedicated device that is lent to a person who has a high risk of stroke according to a medical examination or the like and is used when there is actually suspicion of a stroke.

Server device 200 is a device which is connected to stroke examination device 100 through a network such as the Internet. Here, server device 200 is a storage device for storing information that is used in stroke examination device 100. Server device 200 may be realized by a cloud computer provided on the network, or by an edge computer within the local communication network with which stroke examination device 100 can communicate. In place of server device 200, a storage which stores the same content information may be incorporated in stroke examination device 100. In other words, stroke examination system 500 may be realized by only a single information terminal.

As illustrated in FIG. 2, stroke examination device 100 includes obtainer 101, determiner 102, examiner 103, diagnosis unit 104, sensor unit 105, transmitter and receiver 106, identifier 107, outputter 108, and storage 109. Here, in the present embodiment, obtainer 101, determiner 102, examiner 103, diagnosis unit 104, and identifier 107 are realized by a central processing unit (CPU) and a memory included in controller 110 and a program stored in the memory being executed.

Outputter 108 includes a display unit including a display or the like and an audio output unit including a loudspeaker or the like.

Storage 109 functions as memory which stores a program that is executed by the CPU of controller 110 and profile information and the like relating to brain disorder of the subject.

Obtainer 101 is a processor which obtains profile information relating to brain disorder of the subject. In the present embodiment, the profile information relating to brain disorder of the subject is information that is stored in storage 201 of server device 200. This information is, for example, data relating to electronic health records of the subject or results of medical examinations of the subject. Accordingly, server device 200 is desirably provided in a medical institution. Obtainer 101 performs processes of converting the obtained profile information, decoding encrypted information and the like as necessary to convert the information in a usable format, and outputs the information to determiner 102. In addition, obtainer 101 obtains the physical quantities detected by various sensors included in sensor unit 105, as profile information. Stroke examination system 500 uses the obtained physical quantities in preliminary examinations of a plurality of examination items. Accordingly, obtainer 101 outputs the obtained physical quantities to determiner 102 as results of the preliminary examinations.

Determiner 102 determines the priority of each of a plurality of preset examination items for stroke, based on the profile information output from obtainer 101. Determiner 102 may also determine the priority of each of the examination items, based on statistical information about the relationship between stroke and the lifestyle of the subject (place of residence, gender, age group, degree of physical activity, dietary habits and the like). Specifically, determiner 102 may infer the site and the like of a possible stroke from statistical information based on the lifestyle of the subject, and may determine the base priority of each of the examination items based on the inference result. Here, the base priority thus determined does not reflect the personal profile information of the subject.

Determiner 102 determines the final priority of each of the examination items by calculating the priority from personal profile information of the subject relative to the determined base priority. In the calculation of the final priority, the determiner 102 gives a lower priority to an examination item described below than the other examination items. The examination item to which the lower priority is given is the examination item where constant symptoms have already been developed due to the brain disorder and the like that previously occurred in the subject and there is a possibility that the onset of symptoms based on new signs of a stroke cannot be found in the examination. In addition, determiner 102 gives a higher priority to an examination item where symptoms have already appeared than to the other examination items, based on the result of the preliminary examination included in the profile information.

For example, in the preliminary examination, when the 6-axis sensor included in sensor unit 105 detects, for example, shaking of stroke examination device 100, there is a possibility of Barre’s sign occurring. Hence, determiner 102 gives a higher priority to the examination item for Barre’s sign than the other examination items. Similarly, sensing for a preliminary examination is preset in each examination item. A preliminary examination is conducted before operating instructions for examination are given to the operator or the subject. When constant symptoms have already been developed due to the brain disorder or the like that previously occurred in the subject for an examination item with a priority which has been increased based on the preliminary examination, the priority is decreased. In other words, here, the decrease of priority is dominant over the increase of priority. In such a manner, determiner 102 determines the final priority by at least decreasing or increasing the priority relative to the base priority based on the profile information .

Examiner 103 is a processor which obtains the physical quantities from various sensors included in sensor unit 105, and conducts an examination for signs of stroke of the subject. For respective examination items, sensors and timing for obtaining the physical quantities are preset in examiner 103. Examiner 103 obtains the physical quantity from the sensor corresponding to the examination item to be examined, and outputs the examination result according to the physical quantity. In this way, examiner 103 according to the present embodiment can be considered as a plurality of examiners 103 which conduct examinations of a plurality of examination items.

The examination result that is output from each examiner 103 may be a binary result, such as whether or not the signs of stroke have been detected for a given examination item, or may be a result where the possibility of signs of stroke occurring in a given examination item is expressed in a scale, such as 80%. A comprehensive examination result obtained by the examination results that are output from examiners 103 is output to the diagnosis unit. The comprehensive examination result may be, for example, the average of the scaled expressions, the number of examination items for which signs of stroke were detected, or a binary result which indicates whether or not signs of stroke were found for at least one of the examination items. The output of the examination result from each examiner 103 is obtained by inputting the obtained physical quantity to a trained machine learning model that has been trained using, as training data, at least one of, for example, the physical quantities obtained when signs of stroke were detected, and the physical quantities obtained when signs of stroke were not detected. Accordingly, each of examiners 103 includes a trained machine learning model which has performed optimal learning.

Diagnosis unit 104 is a processor which outputs diagnostic information relating to signs of stroke in the subject based on the examination results. The diagnostic information that is output by diagnosis unit 104 includes, for example, at least one of image information which indicates the examination results or inquiry information which externally outputs the examination results. The image information, which are output from diagnosis unit 104 and indicates the examination results, is displayed, for example, on the screen of a smartphone. It is possible for the operator of stroke examination system 500 to look at the displayed image information and take necessary measures. In addition, the inquiry information, which is output from diagnosis unit 104 and externally outputs the examination results, is sent to, for example, a medical institution through a network or the like without any changes. Subsequently, the medical institution side starts handling the case based on the inquiry information received at the medical institution.

Sensor unit 105 includes a group of various sensors included in stroke examination device 100. Sensor unit 105 includes, for example, a camera, a microphone, a touch panel, a fingerprint sensor, a distance sensor, a global positioning system (GPS), a six-axis sensor (a triaxial acceleration sensor and a triaxial angular velocity sensor), a magnetic sensor, and a luminance sensor.

Transmitter and receiver 106 is a communication module which communicatively connects stroke examination device 100 and an external device through a network. Transmitter and receiver 106 is used, for example, when communication is performed between stroke examination device 100 and server device 200, and when communication is performed between stroke examination device 100 and a receiving device at the medical institution for receiving the inquiry information.

Identifier 107 is a processor which identifies whether or not the operator who operates the stroke examination system is the subject. Identifier 107 performs the identification based on the information input to stroke examination system 500. The operation will be described later.

Storage 201 is a storage device such as a semiconductor memory. Storage 201 stores, for example, information that is extracted from the electronic health records of the subject, and information that is extracted from the results of the medical examinations of the subject.

Transmitter and receiver 202 is a communication module which communicatively connects server device 200 and an external device through a network. Transmitter and receiver 202 is used, for example, when communication is performed between server device 200 and stroke examination device 100.

As illustrated in FIG. 3, a portable terminal device, such as a smartphone, includes, for example, controller 301, display unit 302, storage 303, various sensor group (GPS 304, triaxial sensor 305, triaxial angular velocity sensor 306, proximity sensor 307, magnetic sensor 308, ambient light sensor 309, microphone 310 and the like), camera 311, loudspeaker 312, communicator 313, touch panel 314, fingerprint sensor 315, facial authentication sensor group 316, battery 317, and power supply 318.

Controller 301 is capable of controlling a smartphone integrally, and includes a CPU and a storage element (such as static random-access memory (SRAM)) neither of which are illustrated. Controller 301 corresponds to controller 110 in FIG. 2, and includes at least the functions of obtainer 101, determiner 102, examiner 103, and diagnosis unit 104.

Display unit 302 forms part of outputter 108 in FIG. 2, and performs display based on the information received from controller 301.

Storage 303 stores various data which is used by the operating system (OS), various application programs, and various programs read and executed by controller 301. Moreover, storage 303 may store part or all of the profile information relating to brain disorder of the subject that is information stored in storage 201 of server device 200.

Communicator 313 corresponds to transmitter and receiver 106 in FIG. 2, and controls wireless communication techniques such as long term evolution ((LTE), registered trademark) and the fifth generation mobile communication system (known as 5G). Communicator 313 is connected wirelessly to communication base stations (not illustrated) operated by communication companies, and is connected to the Internet through the communication base stations. Note that how to perform external communication is not the essence of the present application, and various types of portable terminal devices that perform communication via Wi-Fi (registered trademark) or wired LAN are not excluded.

Touch panel 314 is capable of receiving an input to the screen (display unit 302) made by the operator of stroke examination system 500 in FIG. 2, that is, the operator of stroke examination device 100, and sending a signal based on the input to controller 301 (for example, which part on the screen is being touched, or the level of the pressure being applied) .

The various sensor group corresponds to sensor unit 105 in FIG. 2. The various sensor group includes GPS 304 which measures the position of a smartphone on the Earth, triaxial sensor 305 which sets X-axis, Y-axis, and Z-axis to a smartphone and measures the acceleration of each axis, triaxial angular velocity sensor 306 which measures the angular velocity in the rotation direction for each axis set by triaxial sensor 305, proximity sensor 307 which detects a nearby object (for example, a face which is approaching a smartphone), magnetic sensor 308 which detects geomagnetism and indicates the direction, ambient light sensor 309 which detects the brightness in the vicinity of a smartphone, microphone 310 which collects ambient sound and voices, camera 311 which captures an image from the front side or the back side of a smartphone, loudspeaker 312 which produces sound, fingerprint sensor 315 which is used for user authentication and the like, and face authentication sensor group 316 which performs face authentication (in practice, operates as a sensor for face authentication in combination with an infrared camera, a floodlight illuminator, a dot projector, etc. (not illustrated) and the illustrated sensor group (proximity sensor 307, ambient light sensor 309, camera 311)).

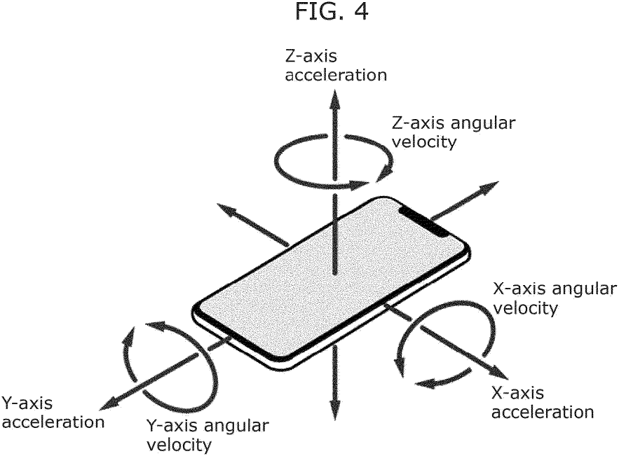

Here, the functions of triaxial sensor 305 (acceleration sensor), and the triaxial angular velocity sensor will be described with reference to FIG. 4. Many smartphones are capable of measuring the acceleration in the three directions of X-axis, Y-axis, and Z-axis (by a triaxial sensor) when the terminal itself is accelerated in the linear direction. Many smartphones are also capable of measuring the acceleration in the direction that the terminal is rotated (expressed as X-axis angular velocity, Y-axis angular velocity, and Z-axis angular velocity, which are also collectively referred to as a triaxial angular velocity).

For example, when the subject captures an image of the face with stroke examination device 100, it is possible to determine at which angle (posture) of stroke examination device 100 the facial image of the subject was captured by using the numerical values detected by these sensors. For example, when stroke examination device 100 is held in a portrait oriented position and a selfie is taken, it is possible to determine whether or not stroke examination device 100 is inclined relative to the horizontal direction, and when inclined, it is possible to determine the inclined angle.

### [Operation]

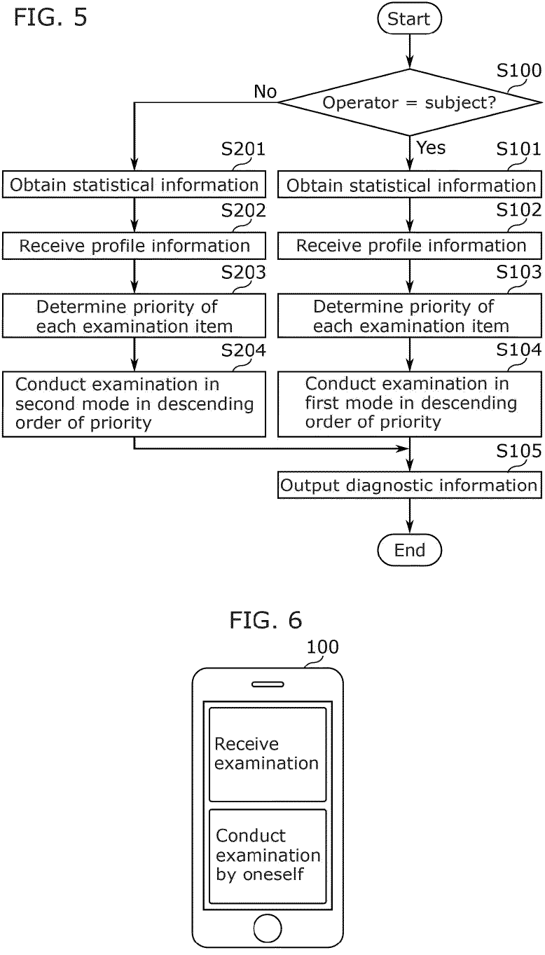

Next, an operation of stroke examination system 500 configured as described above will be described with reference to FIG. 5 to FIG. 7B. FIG. 5 is a flowchart illustrating an example of an operation of the stroke examination system according to the embodiment.

In the present embodiment, even when the operator of stroke examination system 500 is different from the subject, appropriate instructions are output. When an operation of stroke examination system 500 starts, it is first determined whether or not the operator and the subject are the same (S100). FIG. 6 is a first diagram which illustrates one example of an operation screen of the stroke examination system according to the embodiment. FIG. 6 illustrates a state where an image is displayed on the display screen when a program relating to a stroke examination is operated in stroke examination device 100.

As illustrated in FIG. 6, when stroke examination system 500 is operated, a screen is displayed which lets the operator of stroke examination device 100 choose whether the subject is the person who receives an examination (upper part) or the person who conducts an examination by oneself (lower part). Here, when the upper part is chosen, an input indicating that the subject is the person who receives an examination, and that the subject is different from the operator is provided to the system. When the lower part is chosen, an input indicating that the subject is the person who conducts the examination by oneself, and that the subject and the operator are the same is provided to the system. Identifier 107 then performs identification on whether or not the operator and the subject are the same according to these input details. Identifier 107 may perform identification on whether or not the operator and the subject are the same based on an input to a biometric sensor, such as a fingerprint sensor, included in the sensor unit in stroke examination device 100. Identifier 107 may also perform the identification on whether or not the operator and the subject are the same based on an assumption that the owner of the information terminal which implements stroke examination device 100 is the operator. When the operator and the subject are identified as not being the same (No in S100), the processing proceeds to step S201. When the operator and the subject are identified as being the same (Yes in S100), the processing proceeds to step S101. Since step S201 and step S101 are substantially the same operations, step S101 will be described, and description of step S201 will be omitted.

In step S101, obtainer 101 obtains statistical information from an external statistical information server (not illustrated) over the networked through transmitter and receiver 106. The obtained statistical information is output to determiner 102. When step S101 ends, the processing proceeds to step S102, and when step S201 ends, the processing proceeds to step S202. Since step S202 and step S102 are substantially the same operations, step S102 will be described, and description of step S202 will be omitted.

In step S102, obtainer 101 obtains profile information from server device 200 over the network through transmitter and receiver 106, and obtainer 101 receives profile information from sensor unit 105. The obtained profile information is output to determiner 102. When step S102 ends, the processing proceeds to step S103, and when step S202 ends, the processing proceeds to S203. Since step S203 and step S103 are substantially the same operations, step S103 will be described, and description of step S203 will be omitted.

In step S103, determiner 102 determines the priority of each of a plurality of examination items based on the statistical information and the profile information. The method of determining the priority is as described above. When step S103 ends, the processing proceeds to step S104, and when step S203 ends, the processing proceeds to step S204.

In step S104, determiner 102 causes a corresponding one of examiners 103 to operate in descending order of the priority determined. At this time, in step S104, that is, when the operator and the subject are the same, each examination is conducted in a first mode.

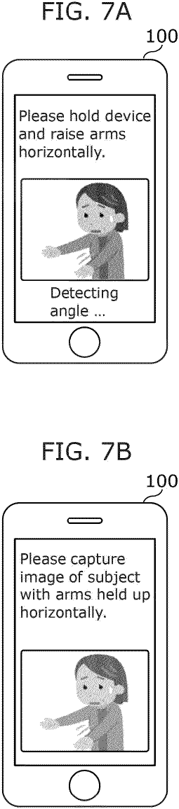

On the other hand, in step S204, determiner 102 causes a corresponding one of examiners 103 to operate in descending order of the priority determined. At this time, in step S204, that is, when the operator and the subject are not the same, each examination is conducted in a second mode. FIG. 7A is a second diagram which illustrates one example of an operation screen of the stroke examination system according to the embodiment. FIG. 7B is a third diagram which illustrates one example of an operation screen of the stroke examination system according to the embodiment. FIG. 7A illustrates a state where an image is displayed on the display screen when an examination of an examination item for Barre’s signs is conducted in the first mode. FIG. 7B illustrates a state where an image is displayed on the display screen when the examination of the examination item for Barre’s signs is conducted in the second mode.

In FIG. 7A, an instruction to hold the terminal (stroke examination device 100) and raise the arms horizontally is being displayed to the operator (who is the same as the subject). Then, the angle of stroke examination device 100 obtained from the six-axis sensor and the like after the instruction is displayed is detected.

On the other hand, in FIG. 7B, an instruction to capture an image of the subject who has arms held up horizontally while holding the terminal (stroke examination device 100) is being displayed to the operator (who is different from the subject). Then, the image obtained from a camera or the like after the instruction is displayed is detected. As described above, different images (instructed operation details) need to be displayed and different sensors need to be used depending on whether the operator or the subject performs the operation. In the present embodiment, different instructions and different sensors can be used appropriately. In the present embodiment, it can also be said that one of a first examiner in the first mode and a second examiner in the second mode is used properly depending on whether or not the operator and the subject are the same.

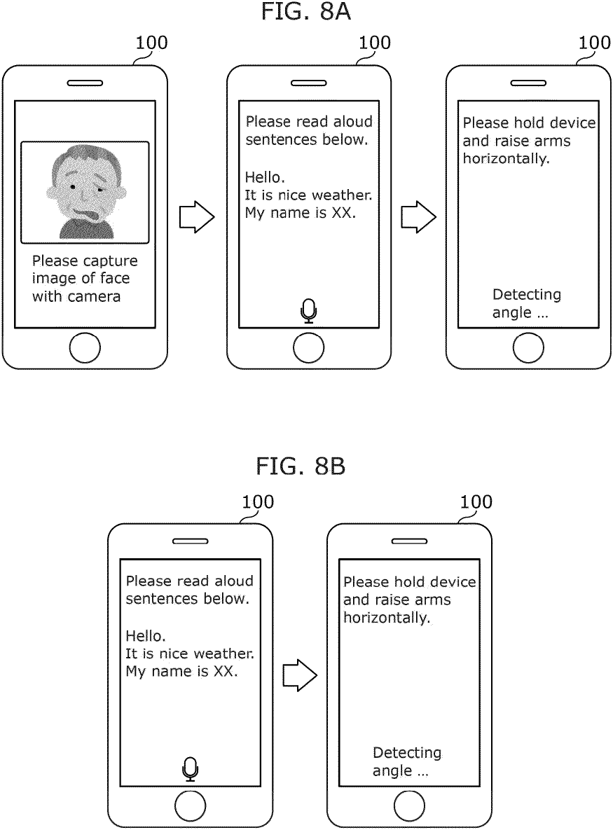

As illustrated in step S104 and step S204, a corresponding one of examiners 103 is operated in the first mode or the second mode in descending order of the determined priority. The examinations for signs of stroke thus conducted are illustrated in FIG. 8A and FIG. 8B. FIG. 8A is a fourth diagram which illustrates an example of an operation screen of the stroke examination system according to the embodiment. FIG. 8B is a fifth diagram which illustrates an example of an operation screen of the stroke examination system according to the embodiment. FIG. 8A illustrates a state where images are displayed on the display screen when examinations of the examination item for the facial paralysis of the subject, the examination item for dysarthria of the subject, and the examination item for Barre’s sign of the subject are conducted sequentially from the left side to the right side in FIG. 8A. FIG. 8B illustrates a state where images are displayed on the display screen when each examination is sequentially executed from the left side to the right side in FIG. 8B in the case where the priority of the examination item for facial paralysis of the subject is set lower than the priorities of the other examination items, and when the priority of examination item for facial paralysis of the subject is lower than or equal to a predetermined value.

FIG. 8A and FIG. 8B illustrate a state where, since the priority of the examination item for the facial paralysis of the subject is decreased, examinations of the other examination items which are the examination item for dysarthria of the subject and the examination item for the Barre’s signs of the subject are conducted first. The priority of the examination item for facial paralysis of the subject is below a predetermined value of the priority at which, rather than conducting the examination of the examination item, the examination should be skipped and a diagnostic result should be output taking into consideration the influence on the time or result. Hence, the examination for facial paralysis itself is not being conducted. In this way, it is possible to speed up the output of the diagnostic result by skipping the examination of the examination item with a low priority as necessary. The predetermined value here may be set experimentally or empirically.

In the present embodiment, an image may be obtained by using the camera as a sensor. Here, obtainment of an appropriate image may fail due to Barre’s signs or the like. For example, there are cases where stroke examination device 100 cannot be held properly so that the orientation of the subject is rotated within the image plane, or stroke examination device 100 cannot be held properly so that the orientation of the subject is rotated within the plane which intersects the image plane. At this time, an image processor (not illustrated) incorporated in examiner 103 may rotate the image and generate a pseudo-normal image. The configuration for such a process will be described later.

In addition, an image as described above may be obtained simply due to being unaccustomed to the operation of stroke examination device 100. In such a case, the displayed message may be changed to cause stroke examination device 100 to be rotated in order that an appropriate image can be obtained. Here, a pseudo-marker (a three-dimensional image whose plane and orientation are clear, such as a coin or a dice) that rotates in conjunction with the 6-axis sensor) may be displayed on the display screen. For example, the above may be realized by giving an instruction to rotate stroke examination device 100 so that the pseudo-marker is oriented appropriately, such as “please rotate the terminal so that the front face of the coin faces forwards”.

Returning to FIG. 5, when step S104 or step S204 ends, the processing proceeds to step S105. In step S105, diagnosis unit 104 outputs diagnostic information, and outputs the diagnostic information to the display screen, an external device and the like.

In such a manner, it is possible to conduct examinations of a plurality of examination items in an appropriate order, and to give an appropriate operating instruction in each examination item based on whether or not the operator and the subject are the same. As a result, it is possible to realize stroke examination system 500 which is capable of quickly providing an examination result with a reduced time loss.

### Regarding Examination for Facial Paralysis of Subject

A method of detecting facial paralysis in an implementation of the embodiment will be described.

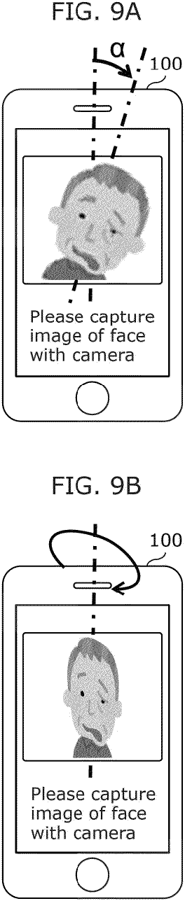

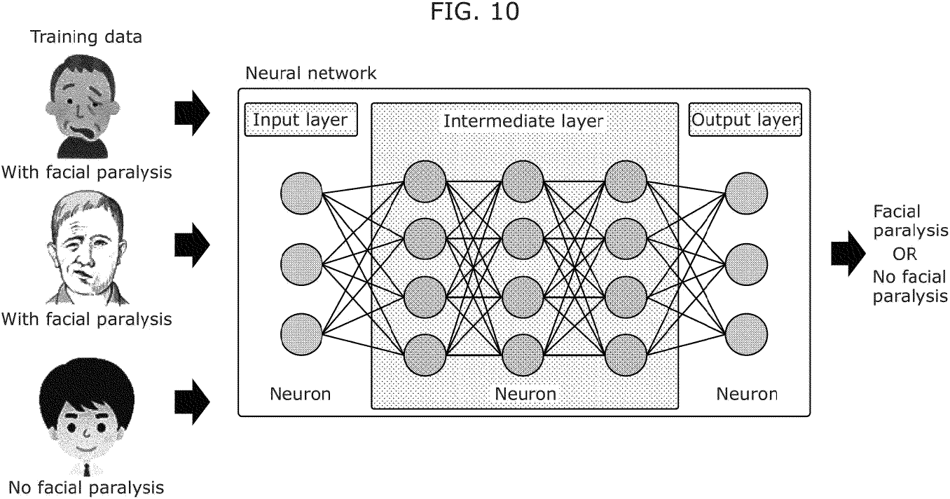

FIG. 9A is a third diagram which illustrates one example of an operation screen of the stroke examination system according to the embodiment. FIG. 9B is a fourth diagram which illustrates one example of an operation screen of the stroke examination system according to the embodiment. FIG. 10 illustrates a method of examining facial paralysis using a neural network that is referred to as deep learning. A data set of a facial image with facial paralysis and corresponding correct information which indicates that there is paralysis, and a data set of a facial image without facial paralysis and corresponding correct information indicating that there is no paralysis are input as training data to a neural network in which the intermediate layer includes multiple stages (in FIG. 10, the first and second faces from the top have paralysis, and the third face does not have paralysis). At this time, the weight coefficient at each node included in the intermediate layer is adjusted to be compatible with the data set by a calculation such as backpropagation.

By performing such an operation, the characteristics of when there is facial paralysis and when there is no facial paralysis are learned by the neural network. When a facial image which has not been used for learning is input to the neural network, determination can be made about whether or not the image has facial paralysis.

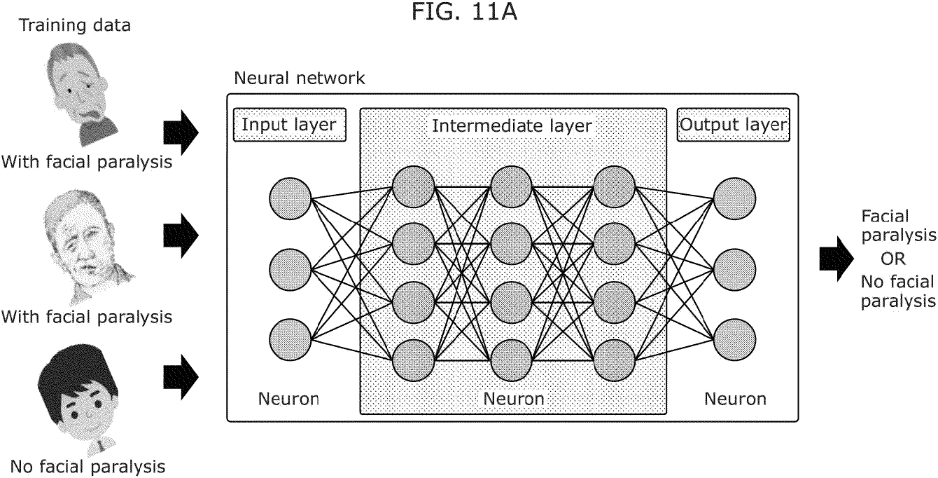

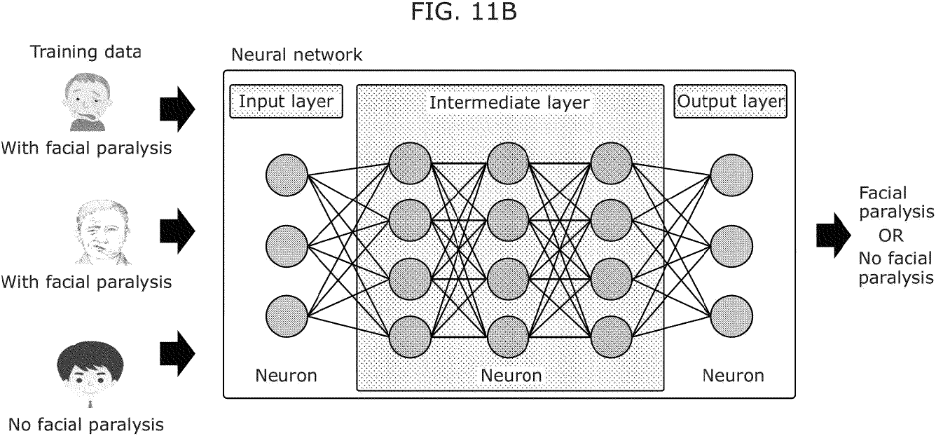

As illustrated in FIG. 10, it is desirable to use forward facing images as training data, when training a neural network. FIG. 11A illustrates one example of a neural network where consideration is given to the influence of rotation of the facial image caused when capturing the image for examining facial paralysis in an implementation of the embodiment. As illustrated in FIG. 11A, the neural network is trained using training data in which the angle is corrected to the angle at which the captured image or video is likely to be misaligned when a stroke patient captures an image of the face (for example, when arm paralysis remains, stroke examination device 100 is rotated either left or right, leading to a rotation of the captured image or video). FIG. 11B illustrates one example of a neural network where consideration is given to the influence of inclination of the upper face (forehead part) and the lower face (chin part) of the facial image caused when capturing the image for examining facial paralysis in an implementation of the embodiment. As illustrated in FIG. 11B, assuming the case where it is difficult to hold stroke examination device 100 in the vertical direction, an image that assumes in advance that the upper face (forehead part) and the lower face (chin part) are inclined back and forth is prepared, and such an image is used as training data for training the neural network.

In such a manner, a plurality of neural networks are generated in advance using facial image data that matches the inclination assumed at the time of capturing. With this, by inputting an image for detection to each of the neural networks when detecting facial paralysis, even when the face in the image is not captured directly from the front, it is possible to sufficiently detect whether or not there is facial paralysis.

In addition, in the example described above, the example has been described where a plurality of neural networks are trained by independently inputting, to each of the neural networks, training data that has been corrected assuming different capturing angles. However, it may be that a single neural network is generated by inputting all training data including the training data after the correction to one neural network.

Although in the description above, the rotation of stroke examination device 100 is corrected using the neural network illustrated in FIG. 11A, the present disclosure is not limited to such an example. From the sensor value output by sensor unit 105 (triaxial sensor 305, triaxial angular velocity sensor 306) of stroke examination device 100 at the time of capturing, the angle at which stroke examination device 100 was inclined with respect to the horizontal direction is obtained. This angle is, for example, angle α illustrated in FIG. 9A as an angular difference between the up and down directions of stroke examination device 100 and the up and down directions of the subject.

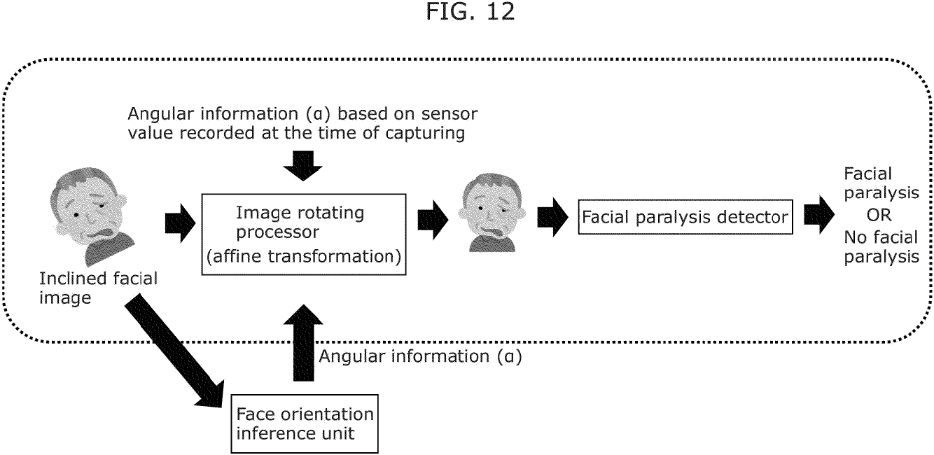

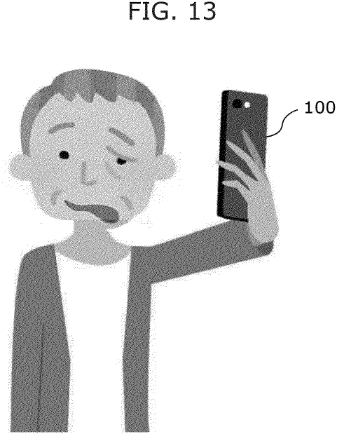

FIG. 12 illustrates one example of processing for correcting the inclination that occurred at the time of capturing the facial image of the subject in an implementation of the embodiment. As illustrated in FIG. 12, angle α may be calculated from the sensor value that is output by sensor unit 105 at the time of capturing, or the sensor value may be stored at the time of capturing the facial image, and angle α may be calculated from the sensor value when the inclined facial image is restored to the original by an image rotating processor (for example, affine transformation). Alternatively, it may be that angle α is calculated from the sensor value at the time of capturing the facial image, and the inclination is corrected by the image rotating processor before recording the facial image in stroke examination device 100 or before inputting the facial image to the facial paralysis detector (neural network capable of detecting the facial paralysis illustrated in FIG. 10). Moreover, the correction processing may be performed by stroke examination device 100, or the image may be transmitted to server device 200 and be corrected by the cloud side. Moreover, the same processing may be performed on, for example, the angular difference between the orientation of the subject in the horizontal plane of stroke inspection device 100 and the orientation of stroke examination device 100 illustrated in FIG. 9B.

### Regarding Examination for Barre’s Signs of Subject

A method for detecting Barre’s signs in an implementation of the embodiment will be described.

When a doctor diagnoses arm paralysis, the subject is instructed to raise both arms horizontally, and a determination is made based on whether or not the subject is able to maintain that state for a predetermined period of time (for example, 5 seconds).

A method of conducting such an examination for Barre’s signs with a terminal device has been proposed (see, for example, PTL 1). In this method, the subject is instructed to maintain raising both arms horizontally, and a moving image of the posture is captured to detect one arm dropping from the wrist position, the elbow position, and the shoulder position.

In the present embodiment, in regards to the horizontal positions of the arms, position information obtained from each of healthy persons and persons who have arm paralysis may be accumulated, so that the neural network is trained with the accumulated information as training data for the healthy persons and the persons which have arm paralysis. Moreover, the determination may be made by simply setting an appropriate threshold value relative to the position of the elbow of the arm and the position of the shoulder or wrist. In this case, the appropriate threshold value indicates how the position of the elbow has changed with respect to the position of the shoulder or wrist, or indicates the position of the wrist dropping from the initial state while the arms are stretched forwards, that is, the posture of outstretching the arms forwards. In any of these cases, the determination can be made by replacing the process of the doctor seeing the patient and making diagnosis with the video image captured by the camera and the image analysis obtained from the video image.

However, with this method, a third party is required as an operator to capture an image of the subject, or a camera needs to be fixed to a predetermined platform or the like in order to capture an image of the subject at an angle of view including the arms of the subject.

Next, a method of detecting Barre’s signs of the subject by using triaxial sensor 305 of stroke examination device 100 will be described. FIG. 13 illustrates a state in which the subject is capturing a facial image of oneself while holding the stroke examination device in an outstretched arm, and conducting examinations for Barre’s signs and facial paralysis simultaneously. As illustrated in FIG. 13, the subject is instructed to hold stroke examination device 100 with one hand or both hands, and to maintain the posture of stretching the one arm or both arms holding stroke examination device 100 in front of the body for a predetermined period of time. At this time, when either the left arm or the right arm is having paralysis, the arm with paralysis drops, and thus, stroke examination device 100 is inclined relative to the horizontal state. The inclined angle may be determined based on a predetermined threshold value. Alternatively, a neural network may be trained by using the sensor values obtained when the same operation is performed by each of healthy persons and persons with arm paralysis as training data, and the detected sensor value may be input to the trained neural network, so that whether or not there is arm paralysis is output.

In addition, when arm paralysis is detected based on whether or not the subject could maintain the posture of holding stroke examination device 100 in one hand or both hands outstretched horizontally for a predetermined period of time, the subject is also viewing the screen of stroke examination device 100 with arms outstretched. Hence, an examination for facial paralysis can also be performed simultaneously by capturing an image of the face of the subject with a camera. By conducting the examinations for Barre’s signs and facial paralysis simultaneously, the examination time taken for TIA (Transient Ischemic Attack) which is one of the signs of stroke is expected to be reduced.

### Regarding Examination for Dysarthria of Subject

In an implementation of the embodiment described above, a method of detecting dysarthria will be described.

The examination for dysarthria is a speech test in the present embodiment, and is an examination for whether or not determined sentences can be spoken smoothly. The voice data obtained when the subject repeatedly utters the prepared sentences is stored in storage 303 via microphone 310. In order to provide a standardized voice sample, a prepared sentence desirably prompts the subject to speak a specific sentence or utter repetitive plosives (“PA”, “KA”, and “TA”) for a specific duration. The quality of utterance of the subject is determined using speech recognition technique. At this time, as pre-processing, detection of voice and other sounds, statistical analysis of voice data, and signal filtering processing for feature extraction may be performed. Raw voice data and/or any derived features are, by way of example, given as inputs to the neural network to perform further feature extraction.

The subject is instructed to read prepared sentences aloud, and dysarthria assessment is performed. The voice data of the subject is input into the neural network. When it is determined as “clear and smooth voice”, then it is determined as normal. When it is determined as “voice was pronounced somewhat inarticulately”, then it is determined as light to medium dysarthria. When it is determined as “voice was pronounced inarticulately to the point of not being understood, or unable to pronounce voice”, then it is determined as severe dysarthria.

### Other Embodiments

Although the embodiment has been described above, the present disclosure is not limited to the above embodiment.

Although examples of the structural elements of the stroke examination system have been described in the above-described embodiment and the like, each function of the structural elements included in the stroke examination system may be distributed to a plurality of portions in the stroke examination system in any manner.

Moreover, in the above embodiment, each structural element may be realized by executing a software program suitable for the structural element. Each structural element may be realized by a program executor, such as a CPU or a processor, reading and executing a software program recorded on a recording medium such as a hard disk or a semiconductor memory.

Moreover, each structural element may be realized by hardware. For example, each structural element may be a circuit (or an integrated circuit). These circuits may form one circuit as a whole, or may be separate circuits. Moreover, each of these circuits may be a general-purpose circuit or a dedicated circuit.

Moreover, the general or specific aspects of the present disclosure may be realized by a system, a device, a method, an integrated circuit, a computer program, or a computer-readable recording medium such as a CD-ROM. Moreover, the general or specific aspects of the present disclosure may be realized by any combination of a system, a device, a method, an integrated circuit, a computer program and a recording medium.

In addition, a form obtained by making various modifications conceivable by those skilled in the art to the embodiment, and a form realized by arbitrarily combining the structural elements and functions in the embodiment and the like without departing from the gist of the present disclosure are also included in the present disclosure.

### [Industrial Applicability]

The present disclosure is useful in the application of conducting an appropriate stroke examination.

1. A stroke examination system which conducts an examination for a sign of stroke in a subject, the stroke examination system comprising:
an obtainer which obtains profile information relating to at least a medical history of a brain disorder of the subject;
a determiner which determines a priority of each of a plurality of examination items for stroke based on the profile information obtained, the determiner giving a lower priority to an examination item where the subject has already developed a constant symptom than an other examination item;
a plurality of examiners which conduct examinations of the plurality of the examination items in a descending order of the priority determined; and
a diagnosis unit which outputs diagnostic information relating to the sign of stroke in the subject based on an examination result obtained by the plurality of examiners.

2. (canceled)

3. The stroke examination system according to claim 1,
wherein the plurality of examination items include at least one of an examination item for facial paralysis of the subject, an examination item for a Barre’s sign of the subject, an examination item for dysarthria of the subject, or an examination item for a gait abnormality of the subject.

4. The stroke examination system according to claim 1,
wherein, when the profile information includes a medical history involving a specific symptom of the subject, the determiner gives a higher priority to an examination item for the specific symptom of the subject among the plurality of examination items than a priority of an other examination item.

5. The stroke examination system according to claim 1,
wherein, when the profile information includes a medical history involving a specific symptom of the subject, the determiner gives a lower priority to an examination item for the specific symptom of the subject among the plurality of examination items than a priority of an other examination item.

6. The stroke examination system according to claim 5,
wherein, when the profile information includes a medical history involving facial paralysis of the subject, the determiner gives a lower priority to an examination item for the facial paralysis of the subject among the plurality of examination items than a priority of an other examination item.

7. The stroke examination system according to claim 5,
wherein, when the profile information includes a medical history involving a Barre’s sign of the subject, the determiner gives a lower priority of an examination item for the Barre’s sign among the plurality of examination items than a priority of an other examination item.

8. The stroke examination system according to claim 5,
wherein, when the profile information includes a medical history involving dysarthria of the subject, the determiner gives a lower priority of an examination item for the dysarthria of the subject among the plurality of examination items than a priority of an other examination item.

9. The stroke examination system according to claim 5,
wherein, when the profile information includes a medical history involving a gait abnormality of the subject, the determiner gives a lower priority of an examination item for the gait abnormality of the subject among the plurality of examination items than a priority of an other examination item.

10. The stroke examination system according to claim 1,
wherein the plurality of examiners include an examiner which does not conduct an examination of an examination item with a priority that is lower than or equal to a predetermined value.

11. The stroke examination system according to claim 1, further comprising
a storage which stores at least one of an onset history of the brain disorder of the subject or medical examination information including information relating to the brain disorder of the subject,
wherein the obtainer obtains, as the profile information, at least one of the onset history or the medical examination information from the storage.

12. The stroke examination system according to claim 1, further comprising
an identifier which identifies the subject and outputs identification information,
wherein the obtainer obtains the profile information corresponding to the identification information output.

13. The stroke examination system according to claim 1,
wherein the profile information further includes information relating to a result of a preliminary examination conducted on the subject.

14. A stroke examination method comprising:
obtaining profile information relating to a medical history of a brain disorder of a subject;
determining a priority of each of a plurality of examination items for stroke based on the profile information obtained so as to give a lower priority to an examination item where the subject has already developed a constant symptom than an other examination item; and
conducting an examination of each of the plurality of examination items in a descending order of the priority determined.

15. A non-transitory computer-readable recording medium for use in a computer, the recording medium having a computer program recorded thereon for causing the computer to execute the stroke examination method according to claim 14.

16. A stroke examination system which conducts an examination for a sign of stroke in a subject, the stroke examination system comprising:
an identifier which identifies whether or not an operator of the stroke examination system is the subject;
an examiner which conducts an examination of a predetermined examination item for stroke, the examiner conducting the examination of the predetermined examination item in a first mode when the operator is identified as being the subject, the first mode being a mode where an operating instruction is given to the subject, the examiner conducting the examination of the predetermined item in a second mode that is different from the first mode when the operator is identified as not being the subject, the second mode being a mode where an operating instruction is given to the operator who is other than the subject; and
a diagnosis unit which outputs diagnostic information relating to the sign of stroke in the subject based on an examination result obtained by the examiner.
